(19) United States
(12) Patent Application Publication
(10) Pub. No.: US 2008/0027246 A1
(43) Pub. Date: Jan. 31, 2008

(54) Process for forming perfluorinated-alkyl sulfonamide substituted norbornene-type monomers

(76) Inventors: Andrew Bell, Lakewood, OH (US); Ramakrishna Ravikiran, Vernon Hills, IL (US); Dietrich Max Fabricius, Hendersonville, NC (US); Peter Wyatt Newsome, Horse Shoe, NC (US)

Correspondence Address: PROMERUS, LLC, 9921 BRECKSVILLE ROAD, BRECKSVILLE, OH, 44141, US

(21) Appl. No.: 11/823,090

(22) Filed: Jun. 26, 2007

Related U.S. Application Data
(60) Provisional application No. 60/833,423, filed on Jul. 26, 2006.

(51) Int. Cl. C07C 311/11

(52) U.S. Cl. 564/96

(57) ABSTRACT

Embodiments in accordance with the present invention are directed to providing processes for forming fluorinated-alkyl and particularly perfluorinated-alkyl sulfonamide substituted norbornene-type monomers.

### BACKGROUND

Perfluorinated-alkyl sulfonamide substituted norbornene-type monomers have considerable promise as repeating units in polymer compositions where alkaline base solubility is a desired characteristic. To that effect, a synthetic route to such monomers that provides high yields of high purity product at a reasonable cost would be desirable. Further, such alternate synthetic route should provide such product having purity, yields and costs equal to or better then the previously known methods that employ sulfonylhalides or anhydrides as a reagent.

### BRIEF DESCRIPTION OF THE DRAWINGS

FIGS. 1, 2, 3 and 4 depict actual GC and NMR data, respectively, for each of exo-/endo-5-(aminomethyl)norbornene (AMNB) and exo-/endo-N-(bicyclo[2.2.1]hept-5-en-2-ylmethyl)-1,1,1-trifluoromethane sulfonamide (TFSNB) prepared in accordance with reaction sequence 1, described below.

### DETAILED DESCRIPTION

The materials of the embodiments of the present invention encompass polycyclic monomers referred to as “norbornene-type monomers.” Such monomers are understood to mean a monomer derived from a substituted or unsubstituted structure such as represented by Structure 1 shown below; where X is —CH₂—, —CH₂CH₂—, O or S and m is an integer from 0 to 5 inclusive:

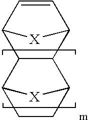

Other than in the operating examples, or where otherwise indicated, all numbers, values and/or expressions referring to quantities of ingredients, reaction conditions, etc., used in the specification and claims are to be understood as modified in all instances by the term “about.”

Various numerical ranges are disclosed in this patent application. Because these ranges are continuous, unless specifically noted otherwise, they include the minimum and maximum values of each range and every value therebetween. Furthermore, unless expressly indicated otherwise, the various numerical ranges specified in this specification and in the claims are approximations that are reflective of the various uncertainties of measurement encountered in obtaining such values.

Some embodiments in accordance with the present invention encompass the preparation of endo/exo-N-(bicyclo[2.2.1]hept-5-en-2-ylmethyl)-1,1,1-trifluoromethane sulfonamide (TFSNB) from endo/exo-aminomethyl norbornene (AMNB). Where such embodiments employ endo/exo AMNB, such is prepared by the reaction of dicyclopentadiene (DCPD) with allyl amine reaction sequence 1, below:

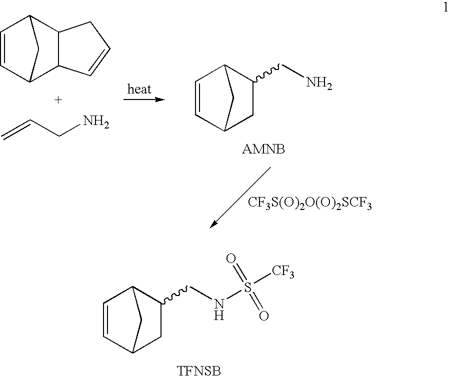

Thus, as per the above sequence, exo/endo AMNB is prepared by a Diels-Alder reaction where DCPD and allyl amine are appropriately combined (about a 1:3 molar ratio) in a Parr Reactor and heated to 210° C. for about 5 hours. The observed pressure range during the reaction is generally from about 260 to 370 psi. After the 5 hours, the reaction mixture is distilled at atmospheric pressure to remove the bulk of the unreacted allyl amine and then the residue is vacuum distilled through a Vigreux column having a distillation head with a splitter to separate out the AMNB product. The AMNB recovered by such vacuum distillation is typically 92-98% pure (by GC analysis) and deemed suitable for conversion to TFSNB.

Preparation of exo/endo-TFSNB in accordance with embodiments of the present invention is typically conducted by charging an appropriately sized reaction vessel with a solution of previously prepared and purified AMNB, triethylamine, and dichloromethane. In a separate vessel, a solution of triflic anhydride and dichloromethane is prepared (approximately 1:1 (v/v)). After cooling the AMNB solution to about −14 to −16° C. and the above triflic anhydride solution added at a rate such that the temperature of the reaction mixture does not exceed 0° C. After the addition is completed, TFSNB product is isolated and purified by first forming the sodium salt and then separating such salt from the organics, dissolving the separated salt in water, washing the aqueous solution with dichloromethane and then acidifying such solution to separate the product as a denser-than-water milky liquid. Subsequent vacuum distillation results in TFSNB as a colorless liquid having a >99% purity (by GC analysis).

It should be noted that the above described Diels-Alder reaction is not limited to the specific materials mentioned. Rather, it is contemplated that one skilled in the art would know that other dienes or other amine-containing dienophiles could be used to form other products that are analogous to the AMNB described above. For example, while the forming of AMNB encompassed the use of allyl amine as the dienophile of the Diels-Alder reaction employed, other dienophiles can also be employed to provide such other products. Thus the use of terminal alkenyl amines in accordance with structure ‘A’, shown below, are within the scope and spirit of embodiments of the present invention.

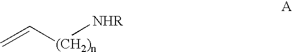

where n is an integer from 1 to 12 and R is hydrogen or a C₁ to C₅ linear or branched alkyl. Thus where a terminal alkenyl amine in accordance with structure ‘A’ and having n greater than 1 is employed, the resulting product would be the appropriately named aminoalkyl norbornene. For example, where n=2, such appropriately named aminoalkyl norbornene would aminoethyl norbornene.

Further, it should also be noted that the forming of the sulfonamide is not limited to mixtures of exo/endo-norbornenes such as the exo/endo-AMNB described above. Rather it is contemplated that essentially pure exo-substituted or endo-substituted aminoalkyl norbornenes could be advantageously employed to eventually form essentially pure exo- or essentially pure endo substituted norbornene fluorosulfonamides. Advantageously, such exo- or endo-substituted aminoalkyl norbornenes are available through either essentially pure exo-norbornene carbonitrile (exo-NBCN), endo-NBCN, or endo or exo-NBCH₂CH₂NH₂ (via exo- or endo-vinylnorbornene (VNB) and its derivatives of endo- or exo-NBCH₂CH₂OH).

With respect to the formation of exo-NBCH₂NH₂ reaction sequence 2, below, is illustrative for the preparation of exo-N-(bicyclo[2.2.1]hept-5-en-2-ylmethyl)-1,1,1-trifluoromethanesulfonamide (Exo-TFSNB) from exo-NBCN. The same reaction sequence is equally applicable where the starting material is endo-NBCN.

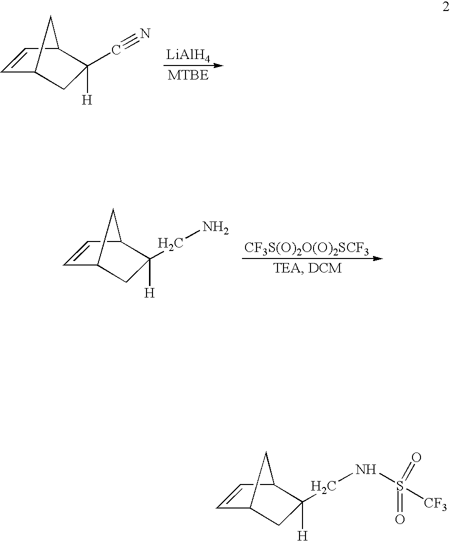

Access to pure endo- or exo-NBCN is accomplished via the addition of acrylonitrile to cyclopentadiene as described in Chemische Berichte (1958), 91, 1516-24. (K. Alder et al.) The ratio of endo to exo adduct from cyclopentadiene and CH₂:CHCN was 3:2. The isomers can be separated by fractional distillation to yielded pure exo-NBCN, boiling point bp_{12 mmHg} 80.5° C., and pure endo-NBCN, bp_{12 mmHg} 88.0° C.

With respect to the formation of exo-NBCH₂CH₂NH₂ reaction sequence 3, below, is illustrative for the preparation of exo-N-(bicyclo[2.2.1]hept-5-en-2-ylethyl)-1,1,1-trifluoromethanesulfonamide (exo-TFSNB) from exo-norbornenecarbonitrile. The same reaction pathway is equally applicable to endo-NBCH₂CH₂NH₂. In the present synthesis scheme, the separation of the exo- and endo-isomers of NBCO₂H can be accomplished by the iodolactonization-reduction of a mixture of endo-/exo-NBCO₂H (for a representative route to exo-NBCO₂H, see L. L. Kiessling et al. in Journal of the American Chemical Society (2005), 127(42), 14536-14537) and for the endo-route see M. Nakazaki, et al. in Journal of Organic Chemistry (1976), 41(7), 1229-33 and Y. Kawanami, in Bulletin of the Chemical Society of Japan (1987), 60(11), 4190-2.

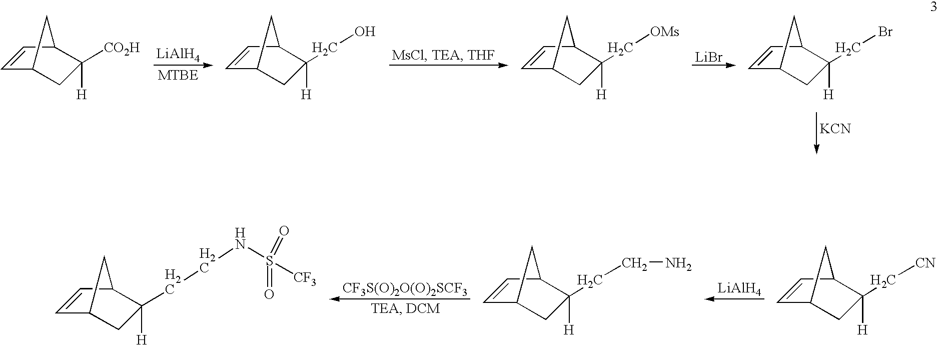

Access to pure endo- or exo-NBCH₂CH₂CH₂NH₂ is accomplished via derivatization of either exo- or endo-NBCH₂CH₂OH. Pure exo- or endo-NBCH₂CH₂OH is generated via the hydroboration of endo/exo-vinylnorbornene by 9-BBN followed by oxidation and separation of the respective isomers via chemical methods, such as iodolactonization, in Y. Inoue. Bulletin of the Chemical Society of Japan (1987), 60(5), 1954-6. Further derivatization to the desired products are accomplished by the established methods described herein. While reaction sequence 4 below describes the exo-synthesis route, this same route is applicable to the endo-diastereomer where the starting material is the appropriate endo isomer.

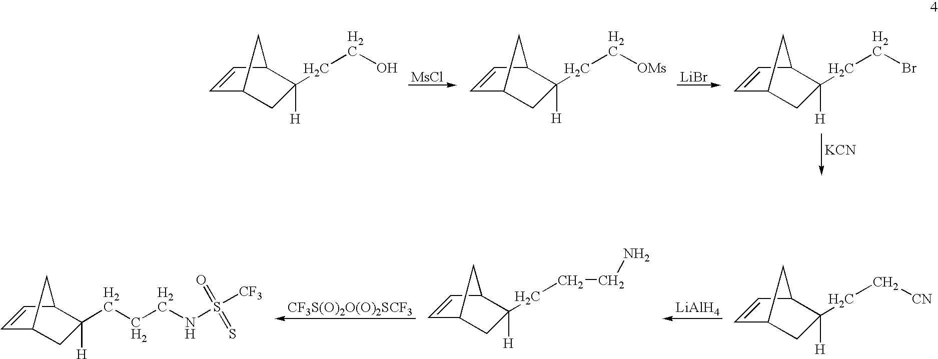

The following examples are provided for illustrative purposes and are not intended to limit the invention in any way.

### EXAMPLES

Example 1 illustrates the formation of first exo-/endo-5-(aminomethyl)norbornene (AMNB) and then exo/endo-N-(bicyclo[2.2.1]hept-5-en-2-ylmethyl)-1,1,1-trifluoromethane sulfonamide (TFSNB) in accordance with reactions sequence 1.

A 19-liter Parr Reactor was charged with 4204 g (31.8 mol) dicyclopentadiene and 6157 g (8.1 liters, 78-96%, 101.8 mol) recycled allylamine and then, while stirring, flushed three times with nitrogen. The reaction was brought to 210° C. over a period of 2 hr 5 min, while the pressure maximized at 360 psi. The reaction was stirred for 5 hours at 210° C. and 271-360 psi and then allowed to cool to room temperature overnight. GC analysis indicated the product mixture to contain, 52.4% exo-/endo-AMNB and 1.7% exo-dicyclopentadiene (DCP). Total weight recovered from the reactor was 10295 g. This material was combined with 8679 g product from an earlier run and distilled under atmospheric pressure through a 14-inch Vigreux column and a distillation head fitted with a splitter to remove the bulk of the unreacted allylamine. Total allylamine recovered was 5674 g. The distillation was continued under vacuum to give the fractions summarized in Table 1. The pot bottoms totaled 3759 g. Total AMNB recovered from this distillation run that was suitable for conversion to TFSNB was, approximately 7435 grams with >92% purity:

In addition, approximately 1400 g of 95-98% purity AMNB was collected, but contained >1% of a high retention time material and was held for further purification The various fractions collected and analytical data for such fractions is provided in Table 1 shown below. The GC analysis for such data was done on a DB5 Column, 30 meters, 0.32 mm ID, 0.25 μm film, 50° C., 5 min hold, then heat to 75° C. at 5° C./min, then to 300° C. @ 25° C./min., Injector temperature: 275° C., Detector temperature: 325° C., Retention time (in minutes): 0.746 (allylamine), 4.866 (exo-DCPD), 5.767 (endo-AMNB), 5.966 (exo-AMNB).

TABLE 1
Vacuum Distillation of Crude AMNB.

| | | | | | | % | | | | total | | | | |
|---|---|---|---|---|---|---|---|---|---|---|---|---|---|---|
| | pot, | head, | vacuum, | | amt, | allyl- | % | % endo | % exo | AMNB | rt >7 | 1.08 | 5.07 | 4.78 |
| fraction | ° C. | ° C. | torr | splitter | g | amine | DCP | AMNB | AMNB | % | m | m | m | m |
| 90PAL32 | | | | | 8679 | 14.4 | 1.3 | 45.3 | 9.3 | 54.6 | 28.2 | | | |
| 90PAL40 | | | | | 10295 | 25 | 1.7 | 43.9 | 8.5 | 52.4 | 19.9 | | | |
| 42-1 | 46-61.7 | 49.7-52 | atm | off | 799 | 97.1 | | | | | | 1.5 | | |
| 42-2 | 62.3-65.2 | 51.2-51.9 | atm | off | 788 | 97.6 | | | | | | 1.7 | | |
| 42-3 | 65.9-67.8 | 51.6 | atm | off | 420 | 99.6 | | 0.3 | | 0.3 | | ns | | |
| 42-4 | 63.8-70.8 | 19.3-53.4 | atm | off | 745 | 99.8 | | 0.16 | | 0.16 | | ns | | |
| 42-5 | 73.2-80.7 | 52.3-52.7 | atm | off | 778 | 99.4 | | 0.6 | | 0.6 | | ns | | |
| 44-6 | 83.7-91.8 | 54.2-52.9 | atm | off | 490 | 98.6 | 0.14 | 0.8 | 0.23 | 1.03 | | ns | | |
| 44-7 | 86.6-85.8 | 52.4-52.6 | atm | off | 203 | 99.2 | 0.11 | 0.62 | | 0.62 | | ns | | |
| | | | | off | | 94.9 | 0.14 | 0.13 | | 0.13 | | 1.8 | | |
| 44-8 | 78.9-92.2 | 27.6-57.0 | atm | off | 582 | 96.1 | 0.21 | 1.3 | | 1.3 | | 1.2 | | |
| 44-9 | 92.7-108.3 | 57.4-65.6 | atm | off | 461 | 96.2 | 0.63 | 2.4 | | 2.4 | | ns | | |
| | | | | on | | 96.4 | 0.62 | 1.1 | | 1.1 | | 1.1 | | |
| 44-10 | 109.2-120.1 | 61.2-69.9 | atm | off | 205 | 95.5 | 1.8 | 1.6 | 0.5 | 2.1 | | 0.41 | | |
| 46-VT | | | vacuum | 20/1 | 203 | 73.8 | 5 | 15.3 | 2.2 | 17.5 | | 1.7 | | |
| 46-11 | 60.1-74.9 | 23.4-52.2 | 20-8.4 | 20/1 | 325.2 | 0.61 | 6.4 | 76.7 | 15.3 | 92 | | 0.15 | 0 | 0.1 |
| 46-12 | 75.2-68.8 | 49.6-49 | 10 | 20/1 | 463.4 | 0.2 | 6.8 | 77.3 | 15.3 | 92.6 | | 0.08 | 0 | 0.1 |
| 46-13 | 68.7-66.8 | 47.7-47.4 | 10-9.2 | 20/1 | 461.7 | 0.13 | 4.5 | 79 | 16.2 | 95.2 | | ni | 0 | 0.1 |
| 46-14 | 67-66.1 | 46.5-46.0 | 8.8-8.4 | 20/1 | 460.4 | 0.11 | 4 | 77.7 | 18.1 | 95.8 | | ni | 0 | 0.1 |
| 46-15 | 66.2-59.4 | 44.7-45 | 7.8-7.7 | 20/1 | 173.12 | 0.09 | 3 | 79.2 | 17.6 | 96.8 | | ni | 0 | 0.1 |
| 46-16 | 64.2-66.1 | 49.9-42.3 | 9.6-6.5 | 20/1 | 768.97 | 0.09 | 2.75 | 79.6 | 17.4 | 97 | | ni | 0 | 0.1 |
| 48-17 | 64.3-65.3 | 39.8-43.4 | 2.12-6.5 | off | 465.37 | 0.06 | 1.6 | 80.5 | 17.7 | 98.2 | 0.02 | 0 | 0 | 0.12 |
| 48-18 | 65.2-67.4 | 35.5-42.6 | 3.2-6.6 | off | 913.9 | 0.06 | 1.7 | 80.8 | 17.1 | 97.9 | 0.03 | 0 | 0 | 0.15 |
| 48-19 | 60-55.3 | 35.2-31.2 | 3.7-2.94 | 20/1 | 459.5 | 0.04 | 1.9 | 79 | 19 | 98 | ni | 0 | 0 | 0.12 |
| | | | | & off | | | | | | | | | | |
| 48-20 | 55.5-62.6 | 28.6-30.9 | 1.64-2.08 | off | 982.9 | 0.05 | 1.4 | 78.3 | 20 | 98.3 | 0.03 | 0 | 0 | 0.19 |
| 48-21 | 62-68.5 | 31.4-33.9 | 2.22-2.58 | off | 1018 | 0.06 | 0.7 | 78.6 | 20 | 98.6 | 0.1 | 0 | 0.15 | 0.21 |
| 48-22 | 68.9-75.5 | 34.0-32.5 | 1.78-2.22 | off | 998.3 | 0.03 | 0.6 | 75.7 | 19.4 | 95.1 | 2.56 | 0 | 1 | 0.27 |
| Fraction 48-22 returned to pot for redistillation | | | | | | | | | | | | | | |
| 48-23 | 55.4-60.5 | 33.2-29.4 | 3.4-1.94 | 20/1 | 474.6 | 0.05 | 1.3 | 79.7 | 18.2 | 97.9 | 0.15 | 0 | 0 | 0.18 |
| 48-24 | 61-67.5 | 28.3-31 | 1.60-2.32 | 20/1 | 467.7 | 0.04 | 0.7 | 77.8 | 20.3 | 98.1 | 0.28 | 0 | 0.51 | 0.24 |
| 48-25 | 67.5-71.6 | 29.8-30.8 | 1.7-2.04 | 30/0.5 | 471 | 0.06 | 0.7 | 75.3 | 22.6 | 97.9 | 1.07 | 0 | 0 | 0.22 |
| | | | | | | 0.04 | 0.8 | 76.6 | 19.1 | 95.7 | 2.31 | 0 | 0.53 | 0.22 |
| | | | | | | ni | 0.54 | 74.9 | 23.3 | 98.2 | 0.72 | 0 | 0.34 | 0.23 |
| 48-26 | 65.6-72.5 | 29.3-31.2 | 2.80-2.46 | 30/0.5 | 468.3 | 0.04 | 0.6 | 75.7 | 21.4 | 97.1 | 1.86 | 0 | 0.1 | 0.3 |
| 48-27 | 73.5-96.7 | 30.3-34.6 | 1.88-2.94 | off | 457.3 | ni | 0.3 | 71.2 | 24.2 | 95.4 | 3.2 | 0 | 0.2 | 0.4 |
| 48-28 | 97-114.1 | 33.5-52.7 | 2.02-2.6 | off | 198.07 | 0.04 | 0.2 | 67.4 | 23.3 | 90.7 | 4.74 | 0 | 0.5 | 0.8 |
| (yellow) | | | | | | | | | | | | | | |

ni = not integrated; ns = no signal

A portion of the aminomethyl norbornene prepared above (92-98%, 3572 g, 27.88 mol), 12 L dichloromethane, and 3381 g (33.47 mol) triethylamine were mixed and chilled to −16° C. with a dry ice-acetonitrile bath. Triflic anhydride (8651 g, 30.66 mol) was split into three portions, each diluted with dichloromethane (totaling 4 L dichloromethane), and each successively added drop wise to the reaction mixture. The temperature was kept between −17° C. to 0° C. and the addition was completed in 5 hrs 34 min. GC analysis indicated all starting material had been consumed. The mixture was cooled to −18° C. and then 3 gal of distilled water was added rapidly, allowing the temperature to rise to 2° C. The mixture was stirred vigorously for 30 minutes as the addition water froze. The mixture was allowed to sit overnight, mixed again the following day and the phases separated. GC analysis indicated the product purity was 61.6%.

The dichloromethane phase was extracted with 3×6 L 10% aqueous NaOH solution. The resulting copious solids were collected by filtration to give 6791 g. The filtrates were chilled in methanol/ice, allowed to sit overnight, and then filtered to collect an additional 3291 g.

The sodium TFSNB was combined with an earlier batch of 10157 g sodium TFSNB and dissolved in 2×12 L of tap water. The solution was filtered. Hot water was generously used to redissolve any crystallized material on the filter. The two yellow solutions were split into 4 batches and each washed with 4 L dichloromethane to remove color, then again with 1-3 L dichloromethane to ensure color removal and/or removal of sludge. Each batch was then acidified with 1000 ml concentrated HCl to pH 0 to 1. The product separates from the aqueous phase as a milky liquid and was collected to give 11261 g (74% yield), >99.5% purity.

The product was dissolved in 8 L dichloromethane to give almost clear yellow solution. This was washed with 2×5 L 10% aqueous sulfuric acid and then separated. The dichloromethane solution was then washed with 2×4 L water. The final wash gave pH=6. The product solution was dried over anhydrous sodium sulfate, decanted and filtered, and then rotary evaporated. The yellow residue was placed in a 12-L flask and vacuum distilled. Colorless product was collected at 101.3-125.5 oC (1.35-3 Torr). Total colorless product collected was 9578 g (63% yield) at >99% purity [86.2:13.8 endo:exo].

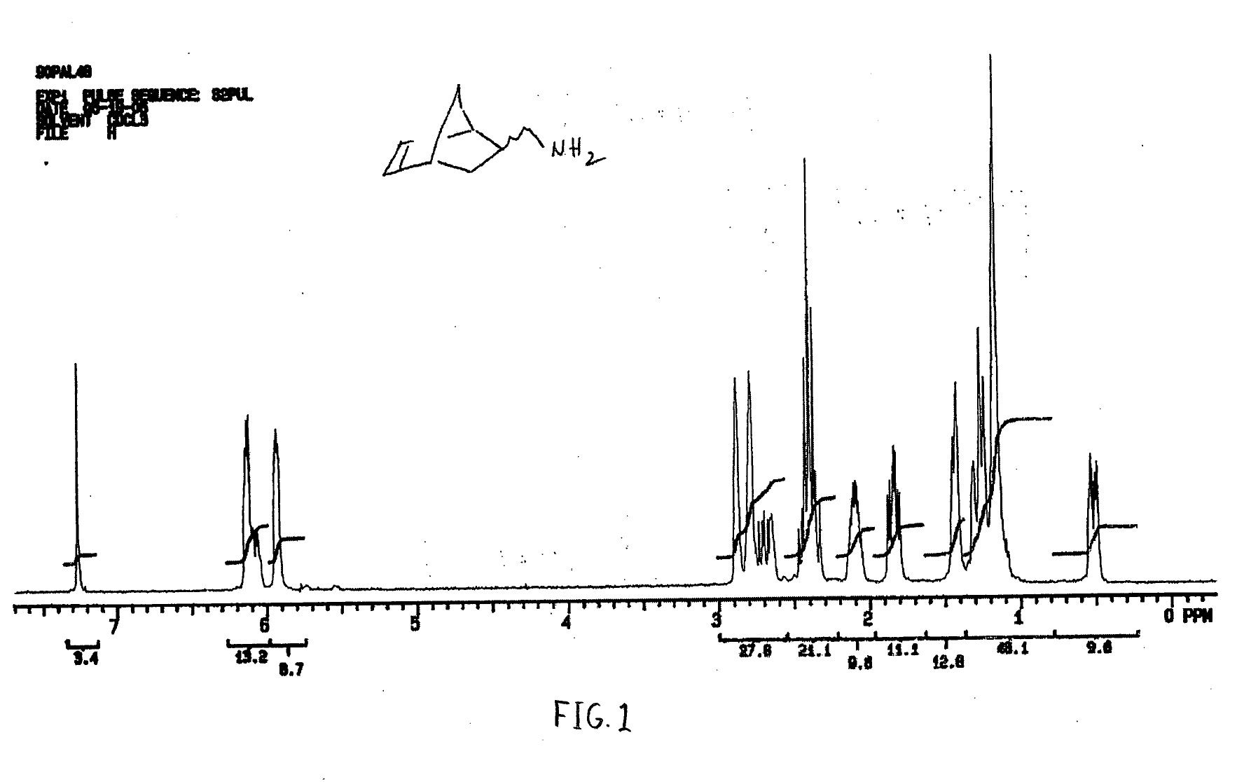

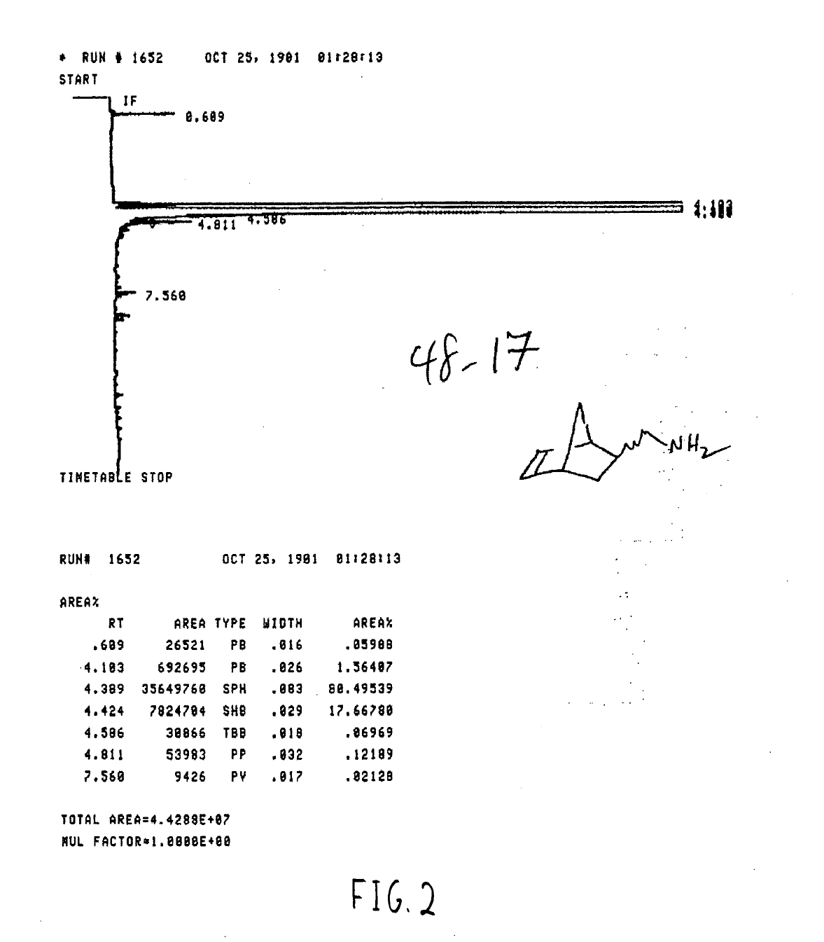

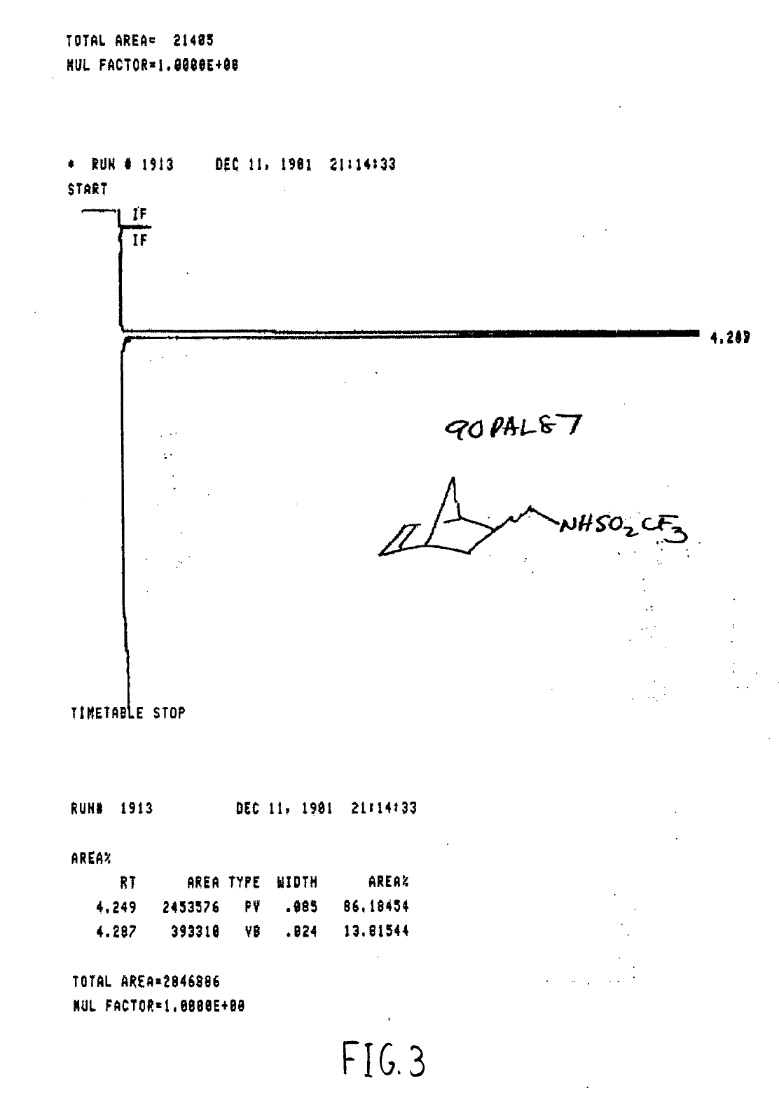

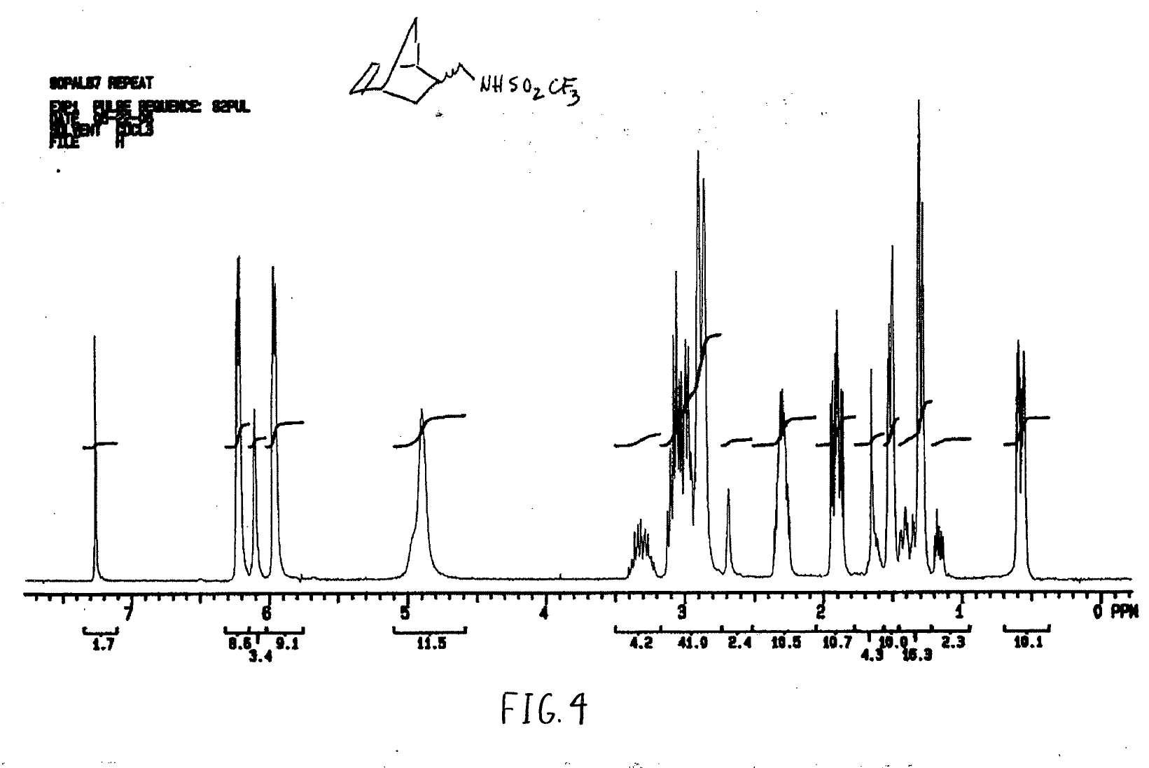

The above GC analysis was done on a DB5 Column, 30 meters, 0.32 mm ID, 0.25 μm film, 75° C. to 300° C. at 15° C./min., Injector temperature: 275° C., Detector temperature: 325° C., Retention time: 4.249 min., 4.287 min. Graphical GC and NMR data are provided for each of the exo-/endo-5-(aminomethyl)norbornene (AMNB) and exo-/endo-TFSNB in FIGS. 1, 2, 3 and 4 that follow.

Example 2 illustrates the preparation of first exo-aminomethylnorbornene by the reduction of the corresponding nitrile and then the forming of exo-TFSNB as shown in reaction sequence 2.

Lithium aluminum hydride (LAH) pellets (239.6 g, 6.3 mol) were mechanically stirred overnight with 3600 ml methyl t-butyl ether (MTBE). The resulting dispersion was chilled to −4.5° C. with a methanol-ice bath before adding exo-norbornenecarbonitrile (339.35 g, 2.85 mol) in 1028 ml MTBE dropwise at a rate to maintain the reaction temperature below 7° C. (Note: the reaction temperature should not be allowed to go below −5° C.; this prevents accumulation of unreacted starting material and a potentially hazardous induction period.) Addition time was 44 minutes. GC analysis showed all starting material had been consumed, 96% product, but 3.2 % intermediate imine still present. The reaction mixture was warmed to 16.2° C. over 60 minutes. GC analysis revealed 98.3% product and only 0.16% imine. The reaction was cooled to −13.8° C. before slowly adding 600 ml distilled water dropwise. The temperature was kept below 23° C. When 280 ml of this initial 600 ml water was added, the reaction mixture thickened to a “lava” stage which prevented good mixing of subsequent added water and allowed pockets of water-saturated lithium and aluminum hydroxides to accumulate. When the mixture thinned again after adding more water, these pockets reacted and generated a sudden exotherm from −2.3° C. to 23° C. After cooling to 20° C., an additional 600 ml MTBE was added to maintain fluidity of the mixture. The addition of initial quench water was complete in 4 h 20 min. Then 1800 ml distilled water was added rapidly to coagulate and precipitate the lithium and aluminum hydroxides and cause separation of the MTBE phases (note: this separation occurred with the first 1450 ml out of the final 1800 ml water). The MTBE phase was decanted and dried over anhydrous sodium sulfate overnight. The dried decant was filtered and rotary evaporated at <30° C. to give 337.07 g (96% yield) of liquid, 99.5% purity by GC. NMR analysis showed the product to still contain approximately 5% MTBE. This was further rotary evaporated to lower the MTBE content to 4% (by NMR), leaving 328.56 g (94% yield). The lithium and aluminum hydroxide residues were covered with 2000 ml MTBE and allowed to sit overnight. The MTBE was decanted, dried over anhydrous sodium sulfate, and rotary evaporated to give 24.86 g, 98.6% by GC, but showing 7.65% MTBE content by NMR. Total yield was 353.42 (96% yield, exclusive of MTBE content).

Exo-aminomethylnorbornene (354.7 g, 2.88 mol), 1460 ml dichloromethane, and 353.41 g (3.50 mol) triethylamine were placed in 4-neck 12-L flask and mechanically stirred while cooling to −14.5° C. Triflic anhydride (545 ml, 3.19 mol) was dissolved in 2160 ml dichloromethane and then added dropwise to the reaction mixture, while maintaining the reaction temperature below 0° C. Addition time was 5 h 33 min. GC analysis showed that all starting material had been consumed. The reaction was allowed to cool to −5° C. before adding 1400 ml distilled water in portions to keep the reaction temperature below 3° C. The phases were separated. The organic portion was washed with 2×2000 ml brine until the final wash gave a pH=6. The organic portion was then extracted with 3×2000 ml 10% aqueous NaOH solution (note: the 2nd 10% aq. NaOH extract caused the organic phase to cloud). The combined aq. NaOH extracts were washed twice with 2000 ml dichloromethane and then acidified to pH 0-1 with 1100 ml concentrated hydrochloric acid. Approximately 600 ml of product separates out. This was diluted with 600 mL dichloromethane, washed with 200 ml brine, and dried over anhydrous sodium sulfate overnight. The dried solution was filtered and then rotary evaporated to give 650.68 g (88% yield) of a white, flaky solid, mp 52.9° C. -53.6° C. GC analysis showed 99.8% purity.

The acidified aqueous phase was extracted with 3×1500 ml dichloromethane. The dichloromethane extracts were washed successively with 1000 ml brine and then 2000 ml brine (to give wash pH=6). The extracts were dried with anhydrous sodium sulfate, filtered, and then rotary evaporated to give 13.56 g white solid, 99% purity by GC.

The cloudy initial dichloromethane portion was washed with 3×1500 ml distilled water, until the 3rd wash gave pH ˜10. The aqueous washes were in turned washed with 1000 ml dichloromethane before acidifying to pH=0-1 with 200 ml concentrated hydrochloric acid. The product separated out as a liquid, which was diluted with dichloromethane, washed with brine, dried over anhydrous sodium sulfate, and then rotary evaporated to yield 21.19 g white solid, 99.5% purity by GC.

The aqueous acid phase was extracted with 3×500 ml dichloromethane. The dichloromethane portions were washed with 100 ml brine, dried over anhydrous sodium sulfate, and then rotary evaporated to give 5.00 g white solid, 99.7% purity by GC. Total yield was 682.25 g (93%).

Example 3 illustrates the preparation of first endo-aminomethyl norbornene by the reduction of the corresponding nitrile and then the forming of endo-TFSNB in a manner analogous to reaction scheme 2.

Lithium aluminum hydride (LAH) pellets (314.4 g, 8.27 mol) were mechanically stirred overnight with 4728 ml MTBE. Endo-norbornenecarbo-nitrile (468.9 g, 3.94 mol) was melted and then dissolved in 2700 ml MTBE. The LAH/MTBE dispersion was chilled to −9° C. with an acetonitrile-dry ice bath before adding the endo-norbornenecarbonitrile solution dropwise at a rate to maintain the reaction temperature below −2° C. Addition time was 73 minutes. GC analysis showed all starting material had been consumed, 71% product, but 8 to 16% intermediate imine still present. The reaction mixture was stirred between −7° C. to −3° C. for 5 hr 50 min. GC analysis revealed 98.4% product and <0.7% imine. The reaction was cooled to −25° C. before slowly adding 800 ml distilled water dropwise over 2 hrs. The temperature was kept below 0° C. Then 2300 ml distilled water was added rapidly to coagulate and precipitate the lithium and aluminum hydroxides and cause separation of the MTBE phases (note: this separation occurred with the first 1900 ml out of the final 2300 ml water). The MTBE phase was decanted and dried over anhydrous sodium sulfate overnight. The dried decant was filtered and rotary evaporated at <30° C. to give 486.4 g (>100% yield) of liquid, 98.5% purity by GC. NMR analysis showed the product to still contain approximately 6.5% MTBE.

The lithium and aluminum hydroxide residues were covered with MTBE and allowed to sit overnight. The MTBE was decanted, dried over anhydrous sodium sulfate, and rotary evaporated to give 23.19 g, 97.0% by GC, but showing 22% MTBE content by NMR.

The product crops were combined for Kugelrohr distillation. The first two fractions, totaling 50.58 g, distilled at 40° C. to 50° C. and appeared to contain water. The main fractions distilled at 60° C. to 70° C. to give 318.31 g, 99.7% purity (combined isomers). NMR indicated ˜3.7% exo-isomer while GC indicated 1.9% exo.

A portion of the endo-aminomethylnorbornene prepared above (73.8 g, 0.60 mol), 300 ml dichloromethane, and 72.72 g (0.72 mol) triethylamine were mixed and cooled to −13° C. Triflic anhydride (186.2 g, 0.66 mol) was dissolved in 454 ml dichloromethane and then added dropwise to the reaction mixture, while maintaining the reaction temperature below −2° C. Addition time was 3 hrs. GC analysis showed that all starting material had been consumed. The reaction was allowed to cool to −6° C. Distilled water (300 ml) was added while keeping the reaction temperature below −1° C. The phases were separated. The organic portion was washed with 2×300 ml brine until the final wash gave a pH=5. The organic portion was then extracted with 350 ml 10% aqueous NaOH solution, causing precipitation of solids. The mixture was filtered and the organic portion extracted again with 2×300 ml 10% aq. NaOH. The 10% NaOH extracts were combined, washed with dichloromethane, and filtered. Total solids collected were 215.51 g. The solids were stirred and washed with 500 ml dichloromethane, filtered, and then dissolved in 100 ml hot water. The hot solution was filtered and set aside to crystallize. The crystals were collected by filtration to yield 101.90 g (61% yield). The crystals were redissolved in 150 ml hot water, cooled to <20° C., and then acidified with 26 ml concentrated hydrochloric acid. The phases were separated. The cloudy organic phase, 67.62 g, was diluted with 200 ml dichloromethane, washed with 100 ml brine, washed with 125 ml 10% sulfuric acid, and then with 3×100 ml brine until the washings gave pH 4. The dichloromethane solution was dried over anhydrous sodium sulfate, filtered, and rotary evaporated to give 65.68 g (43% yield) clear, yellow liquid, 99.4% purity (GC). NMR indicated ˜4.4% exo isomer.

The aqueous acid phase was extracted with 3×100 ml dichloromethane. The dichloromethane extracts were combined and washed with brine until the washings gave pH 6. The extract was dried over anhydrous sodium sulfate, filtered, and rotary evaporated to give 8.74 g, 99.77% purity (GC), 5.6% exo (NMR).

The mother liquor from the above crystallization was acidified with 24 ml concentrated hydrochloric acid to pH 0 to 1. The layers were separated and the organic phase (46.80 g) was diluted to 200 ml with dichloromethane. This was washed with 3×100 ml brine until the washings gave pH 6. The dichloromethane solution was dried over sodium sulfate, filtered, and rotary evaporated to give 44.06 g, 99.57% purity (GC), 6.3% exo (NMR).

The aqueous acid phase of the mother liquor was extracted with 3×100 ml dichloromethane. The dichloromethane extracts were combined and washed with brine until the washings gave pH 6. The extract was dried over anhydrous sodium sulfate, filtered, and rotary evaporated to give 4.55 g, 99.42% purity (GC), 8.5% exo (NMR).

The 65.68 g, 8.74 g, and 44.06 g crops were combined and dissolved in 200 ml dichloromethane. This was washed with 200 ml 10% sulfuric acid and then exhaustively washed with water until the washings gave pH=5. After drying over sodium sulfate overnight, the solution was filtered and exhaustively rotary evaporated to recover 108.13 g (71% yield) yellow liquid, 99.8% purity (GC).

Example 4 illustrates the preparation of first exo-cyanomethylnorbornene from the corresponding exo-bromomethylnorbornene, then the reduction of the nitrile to form exo-aminoethylnorbornene followed by the forming of exo-(aminoethyl)norbornene (TFSENB) as shown in reaction sequence 3.

Exo-(bromomethyl)norbornene, (100 g, 0.535 mol), 357 ml DMSO, and 48.73 g KCN (0.749 mol) were mechanically stirred while being heated between 77.4-84.5° C. overnight. GC analysis indicated the presence of approximately 22% starting material and 78% product. The reaction mixture was cooled to room temperature and the undissolved and unreacted KCN filtered away. NaCN (10.40 g, 0.21 mol) was added and then the reaction was reheated to 80.5° C. to 85.2° C. Most of the NaCN dissolved. After 4 hours, GC analysis indicated <1% starting material remained. The reaction was cooled to room temperature and then poured into 2 L distilled water. The mixture was extracted with 3×700 ml MTBE. The MTBE extracts were washed with 3×1000 ml distilled water to remove DMSO and then dried over anhydrous sodium sulfate. After filtering and rotary evaporation, 64.8 g (91% yield) of a dark yellow liquid was recovered. GC analysis indicated 95.7% purity.

LAH pellets (28.76 g, 0.757 mol) were mechanically stirred overnight with 460 ml MTBE. The resulting dispersion was chilled to −3.2° C. with a methanol-ice bath before adding exo-(cyanomethyl)norbornene (47.64 g, 0.358 mol) in 275 ml MTBE dropwise at a rate to maintain the reaction temperature below 0° C. (Note: the reaction temperature should not be allowed to go below −5° C. to avoid accumulation of unreacted starting material and a potentially hazardous induction period.) Addition time was 63 minutes. The cooling bath was removed and the mixture was then heated to 35.4-38° C. for 1.5 hours when GC analysis indicated no further reaction. The mixture was cooled to −10.2° C. (methanol/ice bath) and 75 ml of distilled water was slowly added over 2 hours 20 minutes, keeping the temperature below 0.6° C. A second 225 ml portion of water was added to precipitate the lithium and aluminum byproducts. After stirring 10 minutes, the lithium and aluminum byproducts were allowed to settle and the MTBE phase was decanted. The lithium and aluminum residues were covered with an additional 300 ml MTBE, then mixed, settled, and the MTBE decanted. The MTBE decants were combined, dried over sodium sulfate, then filtered, and rotary evaporated to give 50.57 g (>100% yield) of yellow liquid. GC analysis indicated 95.6% purity.

The product was diluted with dichloromethane to approximately 200 ml and treated with 100 ml 3M hydrochloric acid. The mixture warms and some dichloromethane boils off. The phases were separated and the remaining organic portion extracted with another 100 ml of 3M hydrochloric acid. The aqueous acid extracts were combined and rinsed together with distilled water, causing crystallization of the hydrochloride salt. The crystals were tediously filtered and washed with dichloromethane to yield 72.56 g white crystals. The crystals were stirred with 500 ml dichloromethane, filtered, and washed with dichloromethane to yield 51.31 g. This was mixed with 300 ml distilled water, heated to dissolve, and filtered. Solid sodium bicarbonate was carefully added until no effervescence occurred, resulting in a solution with pH ˜8. This was extracted with 3×100 ml dichloromethane. The dichloromethane extracts were dried over sodium sulfate, filtered, and rotary evaporated to yield only 5.23 g white solid, 94.7% purity by GC. The aqueous solution (A) was retreated with additional sodium bicarbonate until it became milky (pH=9) and was extracted again with 3×100 ml dichloromethane. The dichloromethane extracts were dried over sodium sulfate, filtered, and rotary evaporated to give 106.93 g white solid. This and the previous 5.23 g were dissolved in water and treated with solid sodium carbonate until pH 10-11 was obtained. This was extracted with dichloromethane. The dichloromethane extracts were washed with brine, then with water (causing an emulsion), and then dried over sodium sulfate overnight. After filtration and rotary evaporation, 22.38 g solid was obtained, 98.3% purity by GC.

Exo-(aminoethyl)norbornene (12.36 g, 0.09 mol), 45 ml dichloromethane, and 10.92 g triethylamine (0.108 mol) were mixed and chilled to −11.3° C. Triflic anhydride (27.93 g, 0.099 mol) in 70 ml dichloromethane was added dropwise over 90 minutes while keeping the reaction temperature between −11.3 to −3.9° C. The mixture was stirred two hours below 1° C. The reaction was chilled to <0° C. before adding 45 ml distilled water. The mixture was vigorously stirred before separating the phases. The organic phase was washed with 75 ml brine. The dichloromethane solution was extracted with 25 ml 10% aqueous sodium hydroxide. The aqueous extract immediately crystallized into a solid mass. The organic portion was washed with an additional 2×25 ml 10% sodium hydroxide, which in turn generated additional solid product. The solids were collected by filtration to yield 26.27 g (>100%). This was stirred with 100 ml dichloromethane, filtered, and washed with dichloromethane. The crystals were then dissolved in ˜50 ml hot distilled water, filtered, and then acidified with concentrated hydrochloric acid to pH=0. The lower, organic phase was separated, diluted to ˜40 ml with dichloromethane, washed with 25 ml 10% sulfuric acid, washed with 6×25 ml distilled water and 25 ml brine (to break the emulsion) until the washings gave pH 5. The organic portions were dried over anhydrous sodium sulfate, filtered, and rotary evaporated to give 11.82 g clear, yellow liquid. Purity was 99.6%.

It should be noted that while some embodiments in accordance with the present invention employ a Diels-Alder reaction for forming aminomethyl norbornene, other aminoalkyl norbornenes can be readily prepared. As one of ordinary skill in the art will know, the reaction of allylamine with dicyclopentadiene is analogous to that of the analogous unsubstituted alkenes with dicyclopentadiene since the more electronegative nitrogen atom of the amines is sufficiently spaced from the double bond to make any effect it might have negligible. Thus the inventors believe that the comparative alkene/DCPD reactions shown below are indicative of the results that would be obtained for analogous amine-substituted alkenes.

Comparative Alkene—DCPD Diels-Alder Reactions

For each of the results shown below, DCPD is melted and a weighed amount charged to a premix vessel. The specific alkene was next weighed and added to the premix vessel. The premix vessel was then shaken well. Specific quantities of each chemical charged are listed below.

The specific premix blend was then charged to a reactor. The reactor sparged with nitrogen while being agitated. The reactor was then isolated from the source nitrogen and vent and heated to the reaction temperature shown. Upon reaching the reaction temperature, the agitated reactor was held at this temperature for the prescribed length of time. At the end of this time, the reactor was cooled down and sampled. Final reaction assay is determined by GC area %. Final reaction conversion was measured against the theoretical conversion if all DCPD had reacted to form the norbornene monomer, assuming that GC area % is equivalent to weight %. Purification was accomplished by vacuum distillation of the crude material.

Butyl Norbornene

| | | | | | | Final | Final | Final |
|---|---|---|---|---|---|---|---|---|
| | DCPD | 1-Hexene | Reactor | Reaction | Reaction | Reaction | Reaction | Reaction |
| Wt Ratio | Charged | Charged | Loading | Temperature | Time | Pressure | Assay | Conversion |
| Hexene:DCPD | (g) | (g) | (%) | (C.) | (hr) | (psig) | (Area %) | (%) |
| 65:35 | 39.6 | 73.5 | 50 | 240 | 6 | 197 | 37.4% | 47% |

Hexyl Norbornene

| | | | | | | Final | Final | Final |
|---|---|---|---|---|---|---|---|---|
| | DCPD | 1-Octene | Reactor | Reaction | Reaction | Reaction | Reaction | Reaction |
| Wt Ratio | Charged | Charged | Loading | Temperature | Time | Pressure | Assay | Conversion |
| Octene:DCPD | (g) | (g) | (%) | (C.) | (hr) | (psig) | (Area %) | (%) |
| 65:35 | 53.7 | 99.8 | 65 | 240 | 6 | 64 | 42.4% | 45% |

Decyl Norbornene

| | | | | | | Final | Final | Final |
|---|---|---|---|---|---|---|---|---|
| | DCPD | 1-Dodecene | Reactor | Reaction | Reaction | Reaction | Reaction | Reaction |
| Wt Ratio | Charged | Charged | Loading | Temperature | Time | Pressure | Assay | Conversion |
| Dodecene:DCPD | (g) | (g) | (%) | (C.) | (hr) | (psig) | (Area %) | (%) |
| 80:20 | 31.0 | 124.1 | 65 | 240 | 6 | 44 | 38.4% | 54% |

We claim:

1. A process for forming endo/exo-N-(bicyclo[2.2.1]hept-5-en-2-ylalky)-1,1,1-trifluoromethane sulfonamide (TFSNB) comprising:
charging a reaction vessel with a mixture of endo/exo-5-(aminoalkyl)norbornene (AANB) and triethylamine where the alkyl portion of said AANB comprises a C₁ to C₁₂ linear alkyl moiety;
cooling the mixture to at temperature between −14° C. to −16° C.;
diluting triflic anhydride with dichloromethane to form a mixture thereof having a 1:1 by volume ratio; and
adding such diluted triflic anhydride to the cooled mixture, such addition at a rate appropriate for maintaining the mixture at a temperature below 0° C.

2. The TFSNB of claim 1 where the alkyl portion of the AANB comprises a methyl, ethyl, n-propyl or n-butyl moiety.

3. The TFSNB of claim 1 where the alkyl portion of the AANB comprises a methyl moiety.

4. The TFSNB of any of claims 1 through 4 purified by a method comprising, after the completion of the triflic anhydride addition:
adding an appropriate amount of water with agitation to the mixture;
separating the aqueous and organic phases;
treating the separated organic phase with an aqueous sodium hydroxide solution to cause precipitation a sodium salt of TFSNB;
collecting the sodium salt;
redissolving the collected sodium salt in water to form an aqueous solution of the TFSNB salt; and
acidifying the aqueous solution to a pH of 1 or lower to cause the separation of the purified TFSNB.

5. A process for forming essentially pure exo-N-(bicyclo[2.2.1]hept-5-en-2-ylalky)-1,1,1-trifluoromethane sulfonamide (TFSNB) comprising:
charging a reaction vessel with essentially pure exo-5-(aminoalkyl)norbornene (AANB) and triethylamine where the alkyl portion of said AANB comprises a C₁ to C₁₂ linear alkyl moiety;
cooling the mixture to at temperature between −14° C. to −16° C.;
diluting triflic anhydride with dichloromethane to form a mixture thereof having a 1:1 by volume ratio; and
adding such diluted triflic anhydride to the cooled mixture, such addition at a rate appropriate for maintaining the mixture at a temperature below 0° C.
